(19) United States
(12) Patent Application Publication
(10) Pub. No.: US 2024/0335667 A1
(43) Pub. Date: Oct. 10, 2024

(54) EDM MANUFACTURE OF MEDICAL LEAD FIXATION HELIX

(71) Applicant: Cardiac Pacemakers, Inc., St. Paul, MN (US)

(72) Inventors: Raymond David Gessler, III, Roberts, WI (US); Mark Phillip Olson, Saint Anthony, MN (US); Steven Eric Schultz, West Lakeland, MN (US); Bryan Peter Nelson, Cushing, WI (US); Joseph Nelsen, Forest Lake, MN (US); Chad Joseph Zimmerman, Hugo, MN (US); Corey Scott Rambow, Spring Lake Park, MN (US); Michael Eastburn, White Bear Lake, MN (US)

(21) Appl. No.: 18/627,269

(22) Filed: Apr. 4, 2024

Related U.S. Application Data
(60) Provisional application No. 63/494,318, filed on Apr. 5, 2023.

(51) Int. Cl. A61N 1/362; A61N 1/375

(52) U.S. Cl. CPC A61N 1/362; A61N 1/37518

(57) ABSTRACT

An apparatus for use in manufacturing a component of an implantable medical device comprises a fixture comprising a first fixture member having a plurality of semi-circular recesses in a lower surface thereof, and a second fixture member having a plurality of semi-circular recesses in an upper surface thereof, the first and second fixture members being configured to be selectively fastened together via a plurality of fasteners, wherein in an assembled state of the fixture, each of the semi-circular recesses of the first fixture member lower surface is aligned with an opposing semi-circular recess in the second fixture member upper surface to define a plurality of workpiece openings each dimensioned to receive and secure a respective work piece to be machined to the fixture.

### CROSS REFERENCE TO RELATED APPLICATIONS

The present application claims priority to U.S. Provisional Patent Application No. 63/494,318, filed Apr. 5, 2023, the entire disclosure of which is incorporated herein by reference.

### TECHNICAL FIELD

The present invention relates to systems and methods for manufacturing implantable medical lead components, in particular, a fixation helix for an active fixation implantable lead.

### BACKGROUND

While active fixation implantable medical leads for cardiac rhythm management are known in the art, there is a continuing need for new and improved systems and methods for precise, high volume manufacturing of components for such leads.

### SUMMARY

In Example 1, an apparatus for use in manufacturing a component of an implantable medical device, the apparatus comprising a fixture comprising a first fixture member and a second fixture member configured to be selectively fastened together via a plurality of fasteners. The first fixture member has an upper surface and a lower surface opposite the upper surface, the lower surface including a plurality of semi-circular recesses selectively spaced along a length of the lower surface and a plurality of planar surfaces located between adjacent semi-circular recesses, the first fixture member further having a front face and an opposite rear face, the front face and the rear face extending between the upper surface and the lower surface, the first fixture member further including a plurality of fastener holes extending through one or both of the lower surface and the upper surface and each configured to receive one of the plurality of fasteners. The second fixture member has an upper surface and a lower surface opposite the upper surface, the upper surface including a plurality of semi-circular recesses selectively spaced along a length of the lower surface, and a plurality of planar surfaces located between adjacent semi-circular recesses, the second fixture member further having a front face and an opposite rear face, the front face and the rear face extending between the upper surface and the lower surface, the second fixture member further including a plurality of fastener holes extending through one or both of the lower surface and the upper surface and each configured to receive one of the plurality of fasteners. In an assembled state of the fixture, the first and second fixture members are secured together by the plurality of fasteners positioned within the plurality of fastener holes, with the lower surface of the first fixture member abutting the upper surface of the second fixture member, and each of the semi-circular recesses of the first fixture member lower surface is aligned with an opposing semi-circular recess in the second fixture member upper surface to define a plurality of workpiece openings each dimensioned to receive and secure a respective work piece to be machined to the fixture.

In Example 2, the apparatus of Example 1, wherein in the assembled state the front faces of the first fixture member and the second fixture member are aligned to define a front face of the fixture.

In Example 3, the apparatus of Example 2, wherein in the assembled state the rear faces of the first fixture member and the second fixture member are aligned to define a rear face of the fixture.

In Example 4, the apparatus of Example 3, wherein the workpiece openings extend through the front and rear faces of the fixture.

In Example 5, the apparatus of any of Examples 1-4, wherein one or both of the first fixture member and the second fixture member includes a plurality of attachment holes extending through the upper and lower surfaces thereof to receive attachment elements for securing the fixture to a wire electrical discharge machine.

In Example 6, the apparatus of any of Examples 1-5, further comprising one or more alignment members, wherein the first fixture member and the second fixture member each includes a plurality of alignment holes sized to receive one of the alignment members to align the first fixture member with the second fixture member in the assembled state.

In Example 7, the apparatus of any of Examples 1-6, further comprising a loading base having an upper planar surface, and a plurality of workpiece supports extending from the upper planar surface and located to align with and extend through the workpiece openings in the fixture assembly when fixture is operatively positioned over the upper planar surface.

In Example 8, the apparatus of Example 7, wherein each of the workpiece supports is dimensioned so that a workpiece to be machined can be positioned thereover.

In Example 9, a method of machining a fixation helix for an implantable active fixation lead, the method comprising providing a loading base having an upper planar surface and a plurality of workpiece supports extending from the upper planar surface, wherein a workpiece is disposed over each of the workpiece supports, each workpiece comprising a wire selectively formed into a helical arrangement and having a proximal end portion positioned adjacent to the upper planar surface and an opposite distal end portion. The method further comprises securing each of the workpieces within a respective through hole of a fixture having a front face and a rear face with the proximal end portion extending proud of the rear face and the distal end portion extending from the front face. Additionally, the method comprises removing the fixture from the assembly base with the workpieces secured to the fixture, mounting the fixture with the workpieces secured thereto within a working chamber of a wire electrical discharge machine, and machining each workpiece using an EDM process to form a finished fixation helix.

In Example 10, the method of Example 9, wherein the fixture includes a first fixture member and a second fixture member, and wherein securing each of the workpieces includes positioning the first fixture member proximate the workpieces and positioning the second fixture member proximate the workpieces opposite the first fixture member and fastening the first fixture member and the second fixture member together with the workpieces disposed therebetween.

In Example 11, the method of Example 10, wherein the first fixture member has an upper surface and a lower surface opposite the upper surface, the lower surface including a plurality of semi-circular recesses selectively spaced along a length of the lower surface and a plurality of planar surfaces located between adjacent semi-circular recesses, the first fixture member further having a front face and an opposite rear face, the front face and the rear face extending between the upper surface and the lower surface. Additionally, the second fixture member has an upper surface and a lower surface opposite the upper surface, the upper surface including a plurality of semi-circular recesses selectively spaced along a length of the lower surface, and a plurality of planar surfaces located between adjacent semi-circular recesses, the first fixture member further having a front face and an opposite rear face, the front face and the rear face extending between the upper surface and the lower surface. Positioning the first fixture member proximate the workpieces and positioning the second fixture member proximate the workpieces opposite the first fixture member includes positioning the first fixture member proximate the workpieces with each workpiece adjacent to one of the semicircular recesses in the lower surface of the first fixture member, and positioning the second fixture member proximate the workpieces with each workpiece adjacent to one of the semicircular recesses in the upper surface of the second fixture member.

In Example 12, the method of Example 11, wherein fastening the first fixture member and the second fixture member together includes applying a force to one or both of the first fixture member and the second fixture member to cause the planar surfaces of the lower and upper surfaces thereof, respectively, to abut each other using a plurality of fasteners to secure the first fixture member and the second fixture member together.

In Example 13, the method of Example 12, wherein positioning the first fixture member proximate the workpieces and positioning the second fixture member proximate the workpieces includes positioning the first fixation member and the second fixation member at locations spaced from the upper planar surface of the assembly base to define a predetermined length by which the proximal end portion of each workpiece stands proud of the rear face of the fixture.

In Example 14, the method of any of Examples 9-13, wherein machining each workpiece includes machining the proximal end portion of each workpiece to form a proximal end of the fixation helix, and machining the distal end portion of each workpiece to form a sharpened distal tip of the fixation helix.

In Example 15, the method of Example 14, wherein machining the proximal end portion of each workpiece includes using an EDM process to machine a flat proximal end of each workplace, the flat proximal end lying in a plane substantially orthogonal to a longitudinal axis of the helical arrangement.

In Example 16, an apparatus for use in manufacturing a component of an implantable medical device, the apparatus comprising a fixture comprising a first fixture member and a second fixture member configured to be selectively fastened together. The first fixture member has an upper surface and a lower surface opposite the upper surface, the lower surface including a plurality of semi-circular recesses selectively spaced along a length of the lower surface and a plurality of planar surfaces located between adjacent semi-circular recesses, the first fixture member further having a front face and an opposite rear face, the front face and the rear face extending between the upper surface and the lower surface, the first fixture member further including a plurality of fastener holes extending through one or both of the lower surface and the upper surface. The second fixture member has an upper surface and a lower surface opposite the upper surface, the upper surface including a plurality of semi-circular recesses selectively spaced along a length of the lower surface, and a plurality of planar surfaces located between adjacent semi-circular recesses, the second fixture member further having a front face and an opposite rear face, the front face and the rear face extending between the upper surface and the lower surface, the second fixture member further including a plurality of fastener holes extending through one or both of the lower surface and the upper surface. In an assembled state of the fixture, the first and second fixture members are secured together with the lower surface of the first fixture member abutting the upper surface of the second fixture member, and each of the semi-circular recesses of the first fixture member lower surface is aligned with an opposing semi-circular recess in the second fixture member upper surface to define a plurality of workpiece openings each dimensioned to receive and secure a respective work piece to be machined to the fixture.

In Example 17, the apparatus of Example 16, wherein in the assembled state the front faces of the first fixture member and the second fixture member are aligned to define a front face of the fixture.

In Example 18, the apparatus of Example 17, wherein in the assembled state the rear faces of the first fixture member and the second fixture member are aligned to define a rear face of the fixture.

In Example 19, the apparatus of Example 18, wherein the workpiece openings extend through the front and rear faces of the fixture.

In Example 20, the apparatus of Example 19, wherein one or both of the first fixture member and the second fixture member includes a plurality of attachment holes extending through the upper and lower surfaces thereof to receive attachment elements for securing the fixture to a wire electrical discharge machine.

In Example 21, the apparatus of any of Examples 1-5, further comprising one or more alignment members, wherein the first fixture member and the second fixture member each includes a plurality of alignment holes sized to receive one of the alignment members to align the first fixture member with the second fixture member in the assembled state.

In Example 22, the apparatus of Example 21, further comprising a loading base having an upper planar surface, and a plurality of workpiece supports extending from the upper planar surface and located to align with and extend through the workpiece openings in the fixture assembly when fixture is operatively positioned over the upper planar surface.

In Example 23, the apparatus of Example 22, wherein each of the workpiece supports is dimensioned so that a workpiece to be machined can be positioned thereover.

In Example 24, a method of machining a fixation helix for an implantable active fixation lead, the method comprising providing a loading base having an upper planar surface and a plurality of workpiece supports extending from the upper planar surface, wherein a workpiece is disposed over each of the workpiece supports, each workpiece comprising helical wire and having a proximal end portion positioned adjacent to the upper planar surface and an opposite distal end portion. The method further comprises securing each of the workpieces within a respective through hole of a fixture having a front face and a rear face with the proximal end portion extending proud of the rear face and the distal end portion extending from the front face, mounting the fixture with the workpieces secured thereto within a working chamber of a wire electrical discharge machine, and machining each workpiece using an EDM process to form a finished fixation helix.

In Example 25, the method of Example 24, wherein the fixture includes a first fixture member and a second fixture member, and wherein securing each of the workpieces includes positioning the first fixture member proximate the workpieces and positioning the second fixture member proximate the workpieces opposite the first fixture member and fastening the first fixture member and the second fixture member together with the workpieces disposed therebetween.

In Example 26, the method of Example 25, wherein the first fixture member has an upper surface and a lower surface opposite the upper surface, the lower surface including a plurality of semi-circular recesses selectively spaced along a length of the lower surface and a plurality of planar surfaces located between adjacent semi-circular recesses, the first fixture member further having a front face and an opposite rear face, the front face and the rear face extending between the upper surface and the lower surface, and the second fixture member has an upper surface and a lower surface opposite the upper surface, the upper surface including a plurality of semi-circular recesses selectively spaced along a length of the lower surface, and a plurality of planar surfaces located between adjacent semi-circular recesses, the first fixture member further having a front face and an opposite rear face, the front face and the rear face extending between the upper surface and the lower surface. Positioning the first fixture member proximate the workpieces and positioning the second fixture member proximate the workpieces opposite the first fixture member includes positioning the first fixture member proximate the workpieces with each workpiece adjacent to one of the semicircular recesses in the lower surface of the first fixture member, and positioning the second fixture member proximate the workpieces with each workpiece adjacent to one of the semicircular recesses in the upper surface of the second fixture member.

In Example 27, the method of Example 26, wherein fastening the first fixture member and the second fixture member together includes applying a force to one or both of the first fixture member and the second fixture member to cause the planar surfaces of the lower and upper surfaces thereof, respectively, to abut each other using a plurality of fasteners to secure the first fixture member and the second fixture member together.

In Example 28, the method of Example 27, wherein positioning the first fixture member proximate the workpieces and positioning the second fixture member proximate the workpieces includes positioning the first fixation member and the second fixation member at locations spaced from the upper planar surface of the assembly base to define a predetermined length by which the proximal end portion of each workpiece stands proud of the rear face of the fixture.

In Example 29, the method of any of Examples 28, wherein machining each workpiece includes machining the proximal end portion of each workpiece to form a proximal end of the fixation helix, and machining the distal end portion of each workpiece to form a sharpened distal tip of the fixation helix.

In Example 30, the method of Example 29, wherein machining the proximal end portion of each workpiece includes using an EDM process to machine a flat proximal end of each workplace, the flat proximal end lying in a plane substantially orthogonal to a longitudinal axis of the helical arrangement.

In Example 31, a fixture for use in manufacturing a fixation helix of an implantable medical device, the fixture comprising a first fixture member having a plurality of semi-circular recesses disposed in and along a lower surface thereof, and a second fixture member having a plurality of semi-circular recesses in an upper surface thereof. The first and second fixture members are configured to be selectively fastened together via a plurality of fasteners, and in an assembled state of the fixture, each of the semi-circular recesses of the first fixture member lower surface is aligned with an opposing semi-circular recess in the second fixture member upper surface to define a plurality of workpiece openings each dimensioned to receive and secure a respective work piece to be machined to the fixture.

In Example 32, the fixture of Example 31, wherein in the assembled state, a front face of the first fixture member and a front face of the second fixture member are aligned to define a front face of the fixture.

In Example 33, the fixture of Example 32, wherein in the assembled state, a rear face of the first fixture member and a rear face of the second fixture member are aligned to define a rear face of the fixture.

In Example 34, the fixture of Example 33, wherein the workpiece openings extend through the front and rear faces of the fixture.

In Example 35, the fixture of Example 34, wherein one or both of the first fixture member and the second fixture member includes a plurality of attachment holes extending through the upper and lower surfaces thereof to receive attachment elements for securing the fixture to a wire electrical discharge machine.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic illustration of an exemplary implantable medical device (IMD) system that can be used in relation to embodiments of the present disclosure.

FIG. 2 is a cross-sectional view of a distal end of an active fixation implantable medical lead of the system of FIG. 1, according to embodiments of the present disclosure.

FIGS. 3A-3F are various views of a fixture, and components thereof, for use in precise, high volume manufacture of fixation helixes for active fixation medical device leads, such as the fixation helix of the lead of FIG. 2, according to embodiments of the present disclosure.

FIG. 4 is a schematic illustration of an exemplary manufacturing application utilizing a plurality of the fixtures of FIGS. 3A-3B for use in a wire electrical discharge machining (EDM) process to provide high volume production of medical lead fixation helixes.

FIGS. 5A-5F illustrate an exemplary manufacturing workflow for high volume manufacture of medical lead fixation helixes via wire EDM using the fixture of FIGS. 3A-3B.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

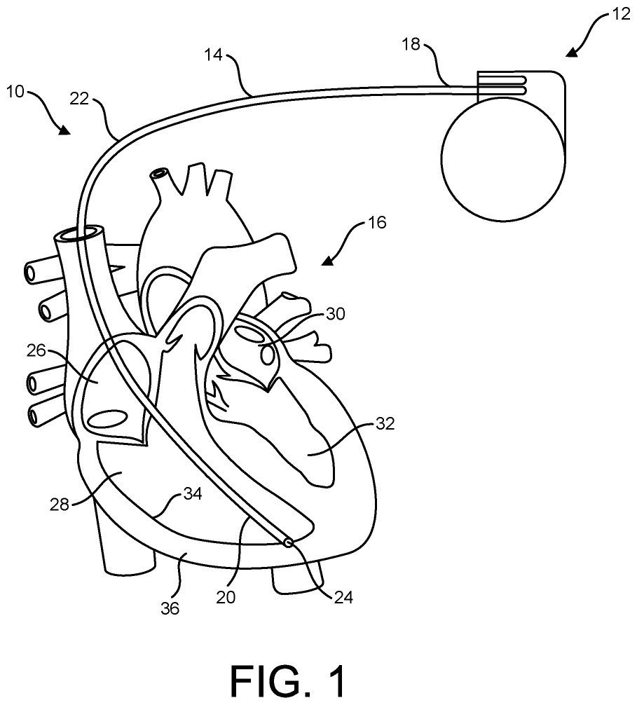

FIG. 1 FIG. 1 is a perspective view of an implantable cardiac rhythm management (CRM) system 10, in accordance with an embodiment. The CRM system 10 includes a pulse generator 12 and a cardiac lead 14. The lead 14 operates to convey electrical signals between the heart 16 and the pulse generator 12. The lead 14 has a proximal region 18 and a distal region 20. The lead 14 includes a lead body 22 extending from the proximal region 18 to the distal region 20. The proximal region 18 is coupled to the pulse generator 12 and the distal region 20 is coupled to the heart 16. The distal region 20 includes a fixation helix 24, which, as will be discussed in greater detail below, locates and/or secures the distal region 20 within the heart 16. As will be explained in detail below, the distal region 20 of the lead 14 includes configurations of the fixation helix 24 that provide improved tissue holding performance.

The pulse generator 12 is typically implanted subcutaneously within an implantation location or pocket in the patient's chest or abdomen. The pulse generator 12 may be any implantable medical device known in the art or later developed, for delivering an electrical therapeutic stimulus to the patient. In various instances, the pulse generator 12 is a pacemaker, an implantable cardioverter/defibrillator (ICD), a cardiac resynchronization (CRT) device configured for bi-ventricular pacing, and/or includes combinations of pacing, CRT, and defibrillation capabilities, e.g., a CRT-D device.

The lead body 22 can be made from any flexible, biocompatible materials suitable for lead construction. In various instances, the lead body 22 is made from a flexible, electrically insulative material. In one embodiment, the lead body 22 is made from silicone rubber. In another embodiment, the lead body 22 is made from polyurethane. In various instances, respective segments of the lead body 22 are made from different materials, so as to tailor the lead body characteristics to its intended clinical and operating environments. In various instances, the proximal and distal ends of the lead body 22 are made from different materials selected to provide desired functionalities.

As shown in FIG. 1, the heart 16 includes a right atrium 26, a right ventricle 28, a left atrium 30 and a left ventricle 32. It can be seen that the heart 16 includes an endothelial inner lining or endocardium 34 covering the myocardium 36. In some instances, as illustrated, the fixation helix 24, located at the distal region 20 of the lead, penetrates through the endocardium 34 and is imbedded within the myocardium 36. In one embodiment, the CRM system 10 includes a plurality of leads 14. For example, it may include a first lead 14 adapted to convey electrical signals between the pulse generator 12 and the right ventricle 28 and a second lead (not shown) adapted to convey electrical signals between the pulse generator 12 and the right atrium 26.

The fixation helix 24 penetrates the endocardium 34 of the right ventricle 28 and is embedded in the myocardium 36 of the heart 16. In some instances, the fixation helix 24 is electrically active and thus operates as a helical electrode for sensing the electrical activity of the heart 16 and/or applying a stimulating pulse to the right ventricle 28. In other instances, the fixation helix 24 is not electrically active. The fixation discussed in detail below may also operate as a helical electrode. Rather, in some instances, other components of the lead 14 are electrically active.

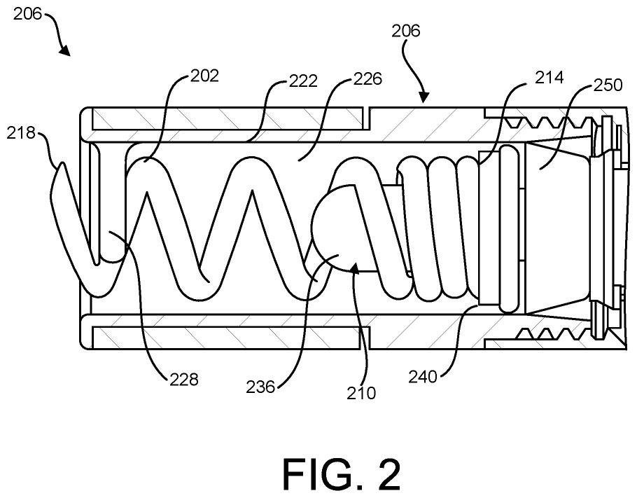

FIG. 2 is a cross-sectional illustration of a distal end portion of an example lead 200 including a fixation helix 202, a housing 206 and a coupler 210 disposed within the housing 206, in accordance with an embodiment of the present disclosure. As shown, the fixation helix 202 has a proximal end 214 and an opposite distal tip 218. As further shown, the housing 206 has an inner surface 222 defining a cavity 226, and a guide member 228 extending radially inward from the inner surface 222 at a distal location of the housing 206. Additionally, in the illustrated embodiment, the coupler 210 has a distal portion 236 and a flange 240 located proximally of the distal end of the coupler 210, having a radial surface. As further shown, the fixation helix 202 is disposed about the distal portion 236 of the coupler 210, with the proximal end 214 of the fixation helix 202 abutting the flange 240. The fixation helix 202 may be mechanically secured to the coupler 210 by any conventional means, e.g., welding, brazing, soldering, swaging, or the like.

As the skilled artisan will appreciate, the coupler 214 is mechanically and electrically coupled to a terminal pin (not shown) at the proximal end of the lead 200 via a conductor member (not shown), such that rotation of the terminal pin also rotates the conductor member, and consequently, the coupler 214 and the fixation helix 202. In the illustrated embodiment, the fixation helix 202 contacts the guide member 228 such that as it rotates, the fixation helix 202 also translates axially relative to the housing 206. It will be appreciated, however, that the illustrated arrangement for providing rotational and translational movement of the fixation helix 202 is only exemplary, and the present disclosure is not intended to be limited to a particular scheme for driving the fixation helix 202 in use. Rather, the present disclosure is applicable to any distal assembly designs, whether now known or later discovered, for use in active fixation implantable medical leads.

Optimal performance and functionality of the active fixation mechanism of the lead 200 in use conditions requires precise manufacturing of the various components, e.g., the fixation helix 202, the housing 206 and the coupler 210. In particular, the interaction between the fixation helix 202 and the guide member 228, as well as the mechanical attachment of the proximal end 214 of the fixation helix 202 to the coupler 210 (e.g., the abutment between the proximal face 214 and the flange 240 in the illustrated embodiment) can be adversely impacted where, for example, the proximal end 214 is not flat and/or is out of plane with a plane that is orthogonal to the longitudinal axis of the fixation helix. Manufacturing techniques conventionally employed for machining helical wires for active fixation helixes may be prone to such imperfections.

The inventors of the present disclosure have discovered that a wire electrical discharge machining (wire EDM) process can be employed for precise machining of the proximal end 214 of the fixation helix 202 (i.e., the surface abutting the flange 240 of the coupler 210) as well as the profile of the distal tip 218 of the fixation helix 202. Briefly, wire EDM, also known as spark machining, spark eroding, die sinking, wire burning or wire erosion, is a metal fabrication process whereby a desired shape is obtained by using electrical discharges to remove from the work piece by a series of rapidly recurring current discharges between two electrodes, separated by a dielectric liquid and subject to an electric voltage. One of the electrodes is called the tool-electrode, or simply the tool or electrode, while the other is called the workpiece-electrode, or workpiece. The process depends upon the tool and work piece not making physical contact as is required for mechanical machining processes, e.g., grinding.

The inventors of the present disclosure have further conceived of a novel system of tooling and manufacturing workflows that facilitate high volume machining of unfinished wire workpieces to simultaneously form multiple finished lead fixation helixes using commercially-available wire EDM equipment.

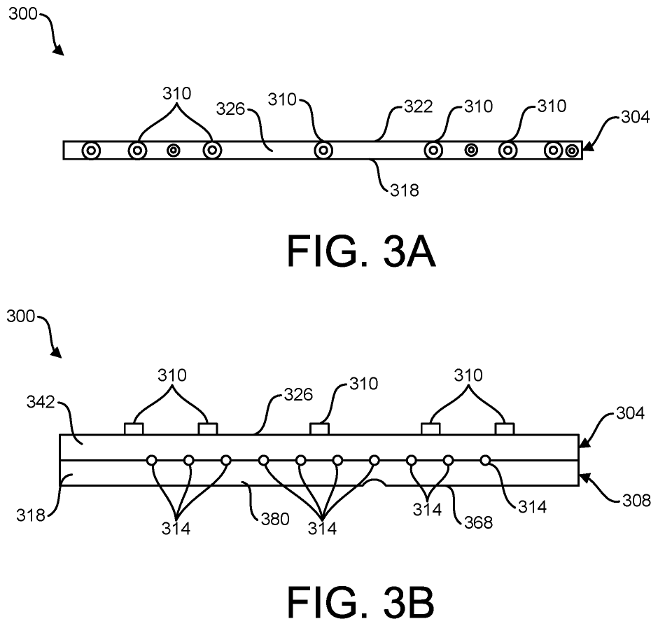

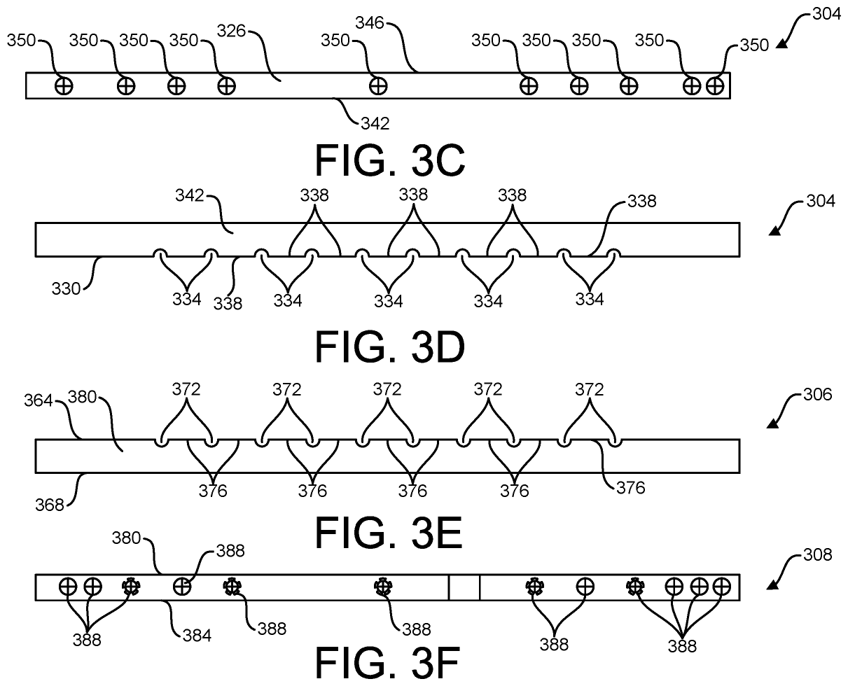

FIGS. 3A-3F are various views of a fixture 300, and components thereof, for use in precise, high volume manufacture of fixation helixes for active fixation medical device leads, such as the fixation helix 202 described above. FIGS. 3A and 3B are top and front plan views, respectively, of the fixture 300 in accordance with embodiments of the disclosure. As shown in FIGS. 3A-3B, the fixture 300 includes a first or upper fixture member 304, a second or lower fixture member 308, a plurality of fasteners 310 securing the upper fixture member 308 and the lower fixture member 308 together, and a plurality of workpiece openings 314. As further illustrated, in the assembled state of the fixture 300, the fixture 300 has a front face 318 and a rear face 322, and the workpiece openings 314 extend through the front and rear faces 318, 322 of the fixture 300. In embodiments, the workpiece openings 314 are each dimensioned to receive and secure to the fixture 300 a workpiece to be subsequently machined, which will be explained in greater detail elsewhere herein.

Referring now to FIGS. 3C and 3D, shown therein are top and front plan views, respectively, of the upper fixture member 304, which as illustrated has an upper surface 326, and a lower surface 330 opposite the upper surface 326. As further shown, the lower surface 330 includes a plurality of semi-circular recesses 334 selectively spaced along the length of the lower surface 330, and a plurality of planar surfaces 338 located between adjacent semi-circular recesses 334. Additionally, the upper fixture member 304 has a front face 342, and an opposite rear face 346, each extending from the upper surface 326 to the lower surface 330, and a plurality of through holes 350 selectively located along and extending through the height of the upper fixture member 304 from the upper surface 326 to the lower surface 330.

Referring now to FIGS. 3E and 3F, shown therein are bottom and front plan views, respectively, of the lower fixture member 308, which as illustrated has an upper surface 364, and a lower surface 368 opposite the upper surface 364. As further shown, the upper surface 364 includes a plurality of semi-circular recesses 372 selectively spaced along the length of the upper surface 364, and a plurality of planar surfaces 376 located between adjacent semi-circular recesses 372. Additionally, the lower fixture member 308 has a front face 380, and an opposite rear face 384, each extending from the upper surface 364 to the lower surface 368, and a plurality of through holes 388 selectively located along and extending through the height of the upper fixture member 304 from the upper surface 364 to the lower surface 368.

Referring back to FIGS. 3A-3B, in the assembled state of the fixture 300, the first and second fixture members 304, 308 are secured together by the plurality of fasteners 310, with the lower surface 330 of the upper fixture member 304 abutting the upper surface 364 of the lower fixture member 308. As shown, each of the semi-circular recesses 334 of the upper fixture member 304 is aligned with an opposing semi-circular recess 372 in the upper surface 364 of the lower fixture member 308 to define one of the workpiece openings 314 in the assembled fixture 300. As further shown, in the assembled state of the fixture 300, the front faces 342, 380 of the first and second fixture members 304, 308, respectively, are aligned to define the front face 318 of the fixture 322. Similarly, the rear faces 346, 384 of the first and second fixture members 304, 308, respectively, are aligned to define the rear face 322 of the fixture 300. Additionally, the workpiece openings 314 extend through the entire depth of the fixture 300 from the front face 318 to the rear face 322.

In embodiments, the through holes 350 and 388 in the upper and lower fixture members 304, 308, respectively are positioned so as to define through holes extending through the entire height of the fixture 300 when assembled. In embodiments, selected ones of the through holes are operable as fastener-receiving holes for receiving the fasteners 310 for securing the first fixture member 304 and the second fixture member 308 together. Selected others of the through holes 350, 388 are operable as attachment holes configured to receive attachment elements, e.g., screws, to secure the assembled fixture 300 to a mounting structure within a working chamber of a wire EDM machine. Still others of the through holes 350, 388 may have additional functionality, e.g., to receive alignment pins to facilitate precise alignment of the upper and lower fixture members 304, 308.

The various embodiments of the disclosure provide a novel fixture arrangement that is especially adapted for high-volume, precise machining of medical lead components, particularly fixation helixes for active fixation implantable medical leads. It is emphasized that the precise number and arrangement of workpiece openings 314 and through holes 350, 388 depicted in the particular embodiment of FIGS. 3A-3F are exemplary only, and are no way intended to limit the scope of the present disclosure. Rather, in embodiments, the specific number and positions of these features, as well as their dimensions and other physical properties, may be varied to accommodate different needs and manufacturing conditions within the scope of this disclosure.

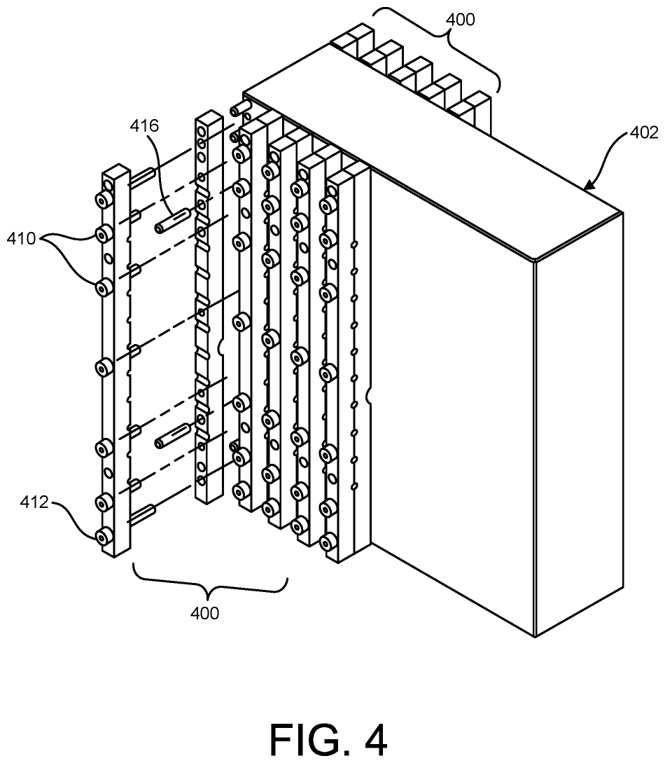

FIG. 4 is a schematic illustration of an exemplary manufacturing application utilizing a plurality of fixtures 400 for use in a wire EDM machine to enable high volume production of medical lead fixation helixes. As will be appreciated, the fixtures 400 may be configured substantially the same as or identical to the fixture 300 described previously, although other configurations may be utilized to accommodate different manufacturing needs. As shown, the embodiments of the present disclosure can facilitate a plurality of fixtures 400 (in the illustrated example, 10) being secured to a mounting structure 402 (shown schematically in FIG. 4) of a wire EDM machine. As further shown, each fixture 400 can be assembled using a plurality of fasteners 410, and can be secured to the mounting structure 402 by one or more attachment elements 412 (e.g., attachment screws). As further shown for illustrative purposes, the fixtures 400 can utilize additional elements, e.g., alignment pins/screws 416 to assist in precise alignment of the fixture components to one another and to the mounting structure 412. In the illustrated example, up to 100 workpieces can be machined to form finished fixation helixes without requiring re-tooling or loading new workpieces into the wire EDM machine.

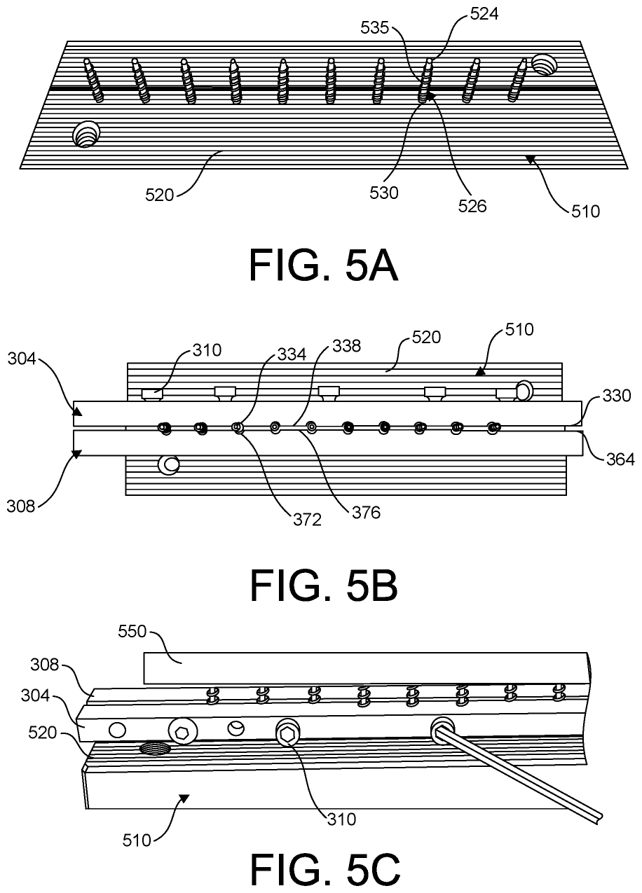

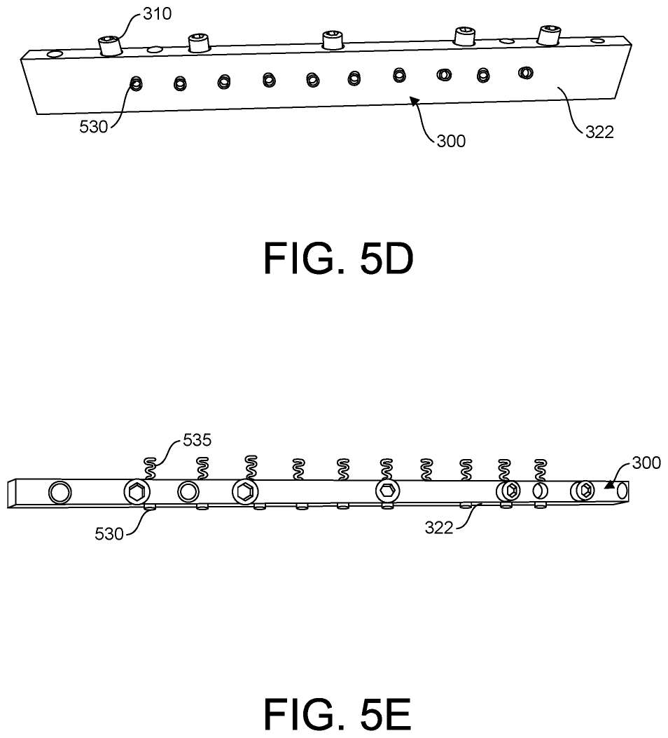

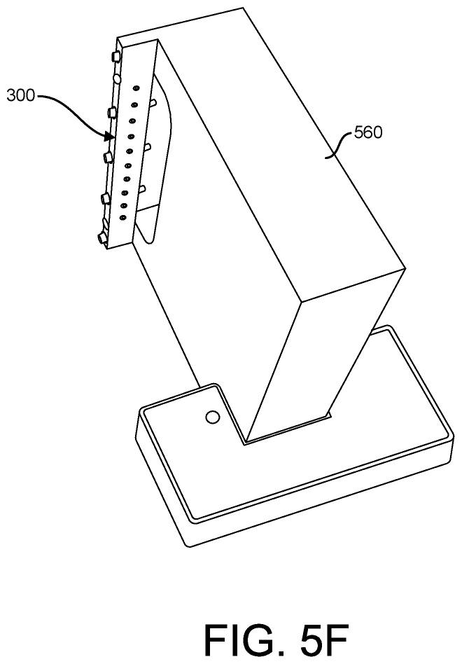

FIGS. 5A-5F illustrate an exemplary manufacturing workflow for high volume manufacture of medical lead fixation helixes via wire EDM using the fixture 300 of FIGS. 3A-3B, according to one exemplary embodiment of the disclosure. To simplify the illustration, only selected elements illustrated in FIGS. 5A-5F are labeled with reference numbers, but the skilled artisan will appreciate that the reference numbers and corresponding description apply equally to all identically configured elements depicted in FIGS. 5A-5F.

Starting with FIG. 5A, a loading base 510 is provided which includes an upper generally planar surface 520 and a plurality of workpiece supports 524 extending from the upper planar surface 520, each being located along the loading base 510 to align with and extend through a respective one of the workpiece openings 314 of the fixture 300. Each of the workpiece supports 524 is dimensioned so that a workpiece to be machined can be positioned thereover. As shown in FIG. 5A, one of the workpieces 526 is disposed over each of the workpiece supports 524. Each workpiece 526 comprises a conductive (e.g., metallic) wire selectively formed into a helical arrangement and having a proximal end portion 530 positioned adjacent to the upper planar surface 520 of the loading base 510, and an opposite distal end portion 535.

As shown in FIG. 5B, the upper fixture member 304 and the lower fixture member 308 are then positioned opposite one another proximate the workpiece supports 524 and, consequently, the workpieces 526 disposed thereon, such that each workpiece support 524 is adjacent to one of the semi-circular recesses 334 in the lower surface 330 of the upper fixture member 304, as well as a corresponding semi-circular recess 372 in the upper surface 364 of the lower fixture member 308. In embodiments, the first fixture member 304 and the second fixture member 308 may be positioned so as to be spaced from the upper planar surface 520 of the loading base 510 (e.g. by spacers or projections extending from the upper planar surface 520) to define a predetermined length by which the proximal end portion 530 stands proud of the rear face 322 (see FIGS. 5D and 5E) of the fixture 300. Alternatively, in embodiments, the upper planar surface 520 may include recesses that can receive the proximal end portion 530 of each workpiece 526 so that it extends below the upper planar surface 520 by a predetermined depth.

As shown in FIG. 5C, the first and second fixture members 304, 308 can then be fastened together with the workpiece supports 524 and corresponding workpieces 526 disposed between them, e.g., by tightening the fasteners 310 with the first and second fixation members 304, 308 still positioned as desired over the upper planar surface 520 of the loading base 510. In the illustrated embodiment, a support jig 550, which includes recesses located to receive the workpiece supports 524, can optionally be utilized to maintain the positions of the workpiece supports 524 during tightening of the fasteners 310.

Complete tightening of the fasteners results in securing the workpieces 526 to the assembled fixture 300 with each workpiece 526 disposed within a respective workpiece opening in the fixture 300, and with the proximal end portion 530 of each workpiece 526 extending proud of the rear face 322 of the fixture 300 and the distal end portion 535 of the workpiece 526 extending from the front face 318 of the fixture 300, and the fixture 300 with the plurality of workpieces 526 secured thereto can then be removed from the loading base 510 by lifting the fixture 300 so that the workpieces 526 slide over their respective workpiece supports 524 (see FIGS. 5D and 5E). As depicted in FIG. 5F, the fixture 300 with the workpieces 526 secured thereto can be mounted to a support structure 560 of a wire EDM machine for subsequent machining of the workpieces 526 as desired. In embodiments, such machining may entail forming the proximal end 214 and sharpened distal tip 218 of a finished fixation helix 202, as illustrated in FIG. 2 and the corresponding description above. As further described above, depending on the design and capability of the particular wire EDM machine being used, additional fixtures 300 with workpieces 526 secured thereto may be similarly mounted to the corresponding wire EDM structure.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An apparatus for use in manufacturing a component of an implantable medical device, the apparatus comprising:
a fixture comprising a first fixture member and a second fixture member configured to be selectively fastened together,
wherein the first fixture member has an upper surface and a lower surface opposite the upper surface, the lower surface including a plurality of semi-circular recesses selectively spaced along a length of the lower surface and a plurality of planar surfaces located between adjacent semi-circular recesses, the first fixture member further having a front face and an opposite rear face, the front face and the rear face extending between the upper surface and the lower surface, the first fixture member further including a plurality of fastener holes extending through one or both of the lower surface and the upper surface, and
wherein the second fixture member has an upper surface and a lower surface opposite the upper surface, the upper surface including a plurality of semi-circular recesses selectively spaced along a length of the lower surface, and a plurality of planar surfaces located between adjacent semi-circular recesses, the second fixture member further having a front face and an opposite rear face, the front face and the rear face extending between the upper surface and the lower surface, the second fixture member further including a plurality of fastener holes extending through one or both of the lower surface and the upper surface,
wherein in an assembled state of the fixture:
the first and second fixture members are secured together with the lower surface of the first fixture member abutting the upper surface of the second fixture member, and
each of the semi-circular recesses of the first fixture member lower surface is aligned with an opposing semi-circular recess in the second fixture member upper surface to define a plurality of workpiece openings each dimensioned to receive and secure a respective work piece to be machined to the fixture.

2. The apparatus of claim 1, wherein in the assembled state the front faces of the first fixture member and the second fixture member are aligned to define a front face of the fixture.

3. The apparatus of claim 2, wherein in the assembled state the rear faces of the first fixture member and the second fixture member are aligned to define a rear face of the fixture.

4. The apparatus of claim 3, wherein the workpiece openings extend through the front and rear faces of the fixture.

5. The apparatus of claim 4, wherein one or both of the first fixture member and the second fixture member includes a plurality of attachment holes extending through the upper and lower surfaces thereof to receive attachment elements for securing the fixture to a wire electrical discharge machine.

6. The apparatus of claim 1, further comprising one or more alignment members, wherein the first fixture member and the second fixture member each includes a plurality of alignment holes sized to receive one of the alignment members to align the first fixture member with the second fixture member in the assembled state.

7. The apparatus of claim 6, further comprising a loading base having an upper planar surface, and a plurality of workpiece supports extending from the upper planar surface and located to align with and extend through the workpiece openings in the fixture assembly when fixture is operatively positioned over the upper planar surface.

8. The apparatus of claim 7, wherein each of the workpiece supports is dimensioned so that a workpiece to be machined can be positioned thereover.

9. A method of machining a fixation helix for an implantable active fixation lead, the method comprising:
providing a loading base having an upper planar surface and a plurality of workpiece supports extending from the upper planar surface, wherein a workpiece is disposed over each of the workpiece supports, each workpiece comprising helical wire and having a proximal end portion positioned adjacent to the upper planar surface and an opposite distal end portion;
securing each of the workpieces within a respective through hole of a fixture having a front face and a rear face with the proximal end portion extending proud of the rear face and the distal end portion extending from the front face;
mounting the fixture with the workpieces secured thereto within a working chamber of a wire electrical discharge machine; and
machining each workpiece using an EDM process to form a finished fixation helix.

10. The method of claim 9, wherein the fixture includes a first fixture member and a second fixture member, and wherein securing each of the workpieces includes positioning the first fixture member proximate the workpieces and positioning the second fixture member proximate the workpieces opposite the first fixture member and fastening the first fixture member and the second fixture member together with the workpieces disposed therebetween.

11. The method of claim 10, wherein:
the first fixture member has an upper surface and a lower surface opposite the upper surface, the lower surface including a plurality of semi-circular recesses selectively spaced along a length of the lower surface and a plurality of planar surfaces located between adjacent semi-circular recesses, the first fixture member further having a front face and an opposite rear face, the front face and the rear face extending between the upper surface and the lower surface; and
the second fixture member has an upper surface and a lower surface opposite the upper surface, the upper surface including a plurality of semi-circular recesses selectively spaced along a length of the lower surface, and a plurality of planar surfaces located between adjacent semi-circular recesses, the first fixture member further having a front face and an opposite rear face, the front face and the rear face extending between the upper surface and the lower surface, and
wherein positioning the first fixture member proximate the workpieces and positioning the second fixture member proximate the workpieces opposite the first fixture member includes positioning the first fixture member proximate the workpieces with each workpiece adjacent to one of the semicircular recesses in the lower surface of the first fixture member, and positioning the second fixture member proximate the workpieces with each workpiece adjacent to one of the semicircular recesses in the upper surface of the second fixture member.

12. The method of claim 11, wherein fastening the first fixture member and the second fixture member together includes applying a force to one or both of the first fixture member and the second fixture member to cause the planar surfaces of the lower and upper surfaces thereof, respectively, to abut each other using a plurality of fasteners to secure the first fixture member and the second fixture member together.

13. The method of claim 12, wherein positioning the first fixture member proximate the workpieces and positioning the second fixture member proximate the workpieces includes positioning the first fixation member and the second fixation member at locations spaced from the upper planar surface of the assembly base to define a predetermined length by which the proximal end portion of each workpiece stands proud of the rear face of the fixture.

14. The method of any of claim 13, wherein machining each workpiece includes machining the proximal end portion of each workpiece to form a proximal end of the fixation helix, and machining the distal end portion of each workpiece to form a sharpened distal tip of the fixation helix.

15. The method of claim 14, wherein machining the proximal end portion of each workpiece includes using an EDM process to machine a flat proximal end of each workplace, the flat proximal end lying in a plane substantially orthogonal to a longitudinal axis of the helical arrangement.

16. A fixture for use in manufacturing a fixation helix of an implantable medical device, the fixture comprising:
a first fixture member having a plurality of semi-circular recesses disposed in and along a lower surface thereof; and
a second fixture member having a plurality of semi-circular recesses in an upper surface thereof,
wherein the first and second fixture members are configured to be selectively fastened together via a plurality of fasteners, and
wherein in an assembled state of the fixture, each of the semi-circular recesses of the first fixture member lower surface is aligned with an opposing semi-circular recess in the second fixture member upper surface to define a plurality of workpiece openings each dimensioned to receive and secure a respective work piece to be machined to the fixture.

17. The fixture of claim 16, wherein in the assembled state, a front face of the first fixture member and a front face of the second fixture member are aligned to define a front face of the fixture.

18. The fixture of claim 17, wherein in the assembled state, a rear face of the first fixture member and a rear face of the second fixture member are aligned to define a rear face of the fixture.

19. The fixture of claim 18, wherein the workpiece openings extend through the front and rear faces of the fixture.

20. The fixture of claim 19, wherein one or both of the first fixture member and the second fixture member includes a plurality of attachment holes extending through the upper and lower surfaces thereof to receive attachment elements for securing the fixture to a wire electrical discharge machine.
